(19) United States
(12) Patent Application Publication
(10) Pub. No.: US 2014/0361095 A1
(43) Pub. Date: Dec. 11, 2014

(54) ULTRASONIC MICROVALVE ARRAY UNIT FOR PRODUCTION OF MIST

(71) Applicant: SCENTCOM LTD, Lehavot Haviva (IL)

(72) Inventor: Yossi Haran, Modi'in (IL)

(21) Appl. No.: 14/369,695

(22) PCT Filed: Jan. 13, 2013
(86) PCT No.: PCT/IL13/50033

§ 371 Date: Jun. 29, 2014

Related U.S. Application Data
(60) Provisional application No. 61/585,771, filed on Jan. 12, 2012.

(51) Int. Cl. B05B 17/00; A61L 9/14

(52) U.S. Cl. CPC B05B 17/0646; A61L 9/14
USPC 239/4; 239/102.2

(57) ABSTRACT

The subject matter discloses an apparatus, comprising an array of micro-valves for dispersing mist, each micro valve of the array of micro-valve comprises a needle and a case; an ultrasonic vibrating element for vibrating the array of micro-valves; a mechanical connector for connecting the needles and cases of the array of micro-valves, thus enabling generation of a secondary movement of the needles relative to the cases while the array of micro-valves vibrates, said secondary movement is generated by the vibration of the ultrasonic vibrating element.

### FIELD OF THE INVENTION

The subject matter relates generally to a method and system for dispersion of scent by ultrasonic device. More specifically, the subject matter relates to dispersion of scented mist.

### BACKGROUND OF THE INVENTION

Various attempts have been made to provide means and methods for liquid dispersion. US Patent Application 20110266359 of Scentcom provides an ultrasonic system for scent production. The liquid delivery system is designed for a portable device, and depends on occasional shaking to facilitate the transportation of the liquid scent from the reservoir to the emitter. An important component of ultrasonic scent dispersion devices is the means by which liquid is delivered to the mist emitter (vibrating mesh). The use of wick to carry scent liquid to the scent-emitting mechanism is well known. U.S. Pat. No. 5,161,646, of Charles C describes a mechanism that includes a container for the scent liquid and a wick for carrying the scent liquid to the heat source, based on the heat derived from a resistor and current flow. In later developments, the scent emitting mechanism by heat is replaced with ultrasonic mechanisms based on vibrating mesh, where the wick is still used for carrying the scent liquid.

Unfortunately, this mechanism is suitable for only a limited range of viscosity of the scent liquid and is not suitable for higher liquid viscosity, due to the reversed nebulization phenomenon. In fact, vibrating mesh systems, and current wick using systems have inherent shortcomings. There is therefore, a long felt unmet need for improved systems for dispersing scented mists into the atmosphere.

### SUMMARY

It is an object of the subject matter to disclose an apparatus, comprising: an array of micro-valves for dispersing mist, each micro valve of the array of micro-valve comprises a needle and a case; an ultrasonic vibrating element for vibrating the array of micro-valves; a mechanical connector for connecting the needles and cases of the array of micro-valves, thus enabling generation of a secondary movement of the needles relative to the cases while the array of micro-valves vibrates, said secondary movement is generated by the vibration of the ultrasonic vibrating element.

In some cases, the mechanical connector results in the needles of the array of micro-valve arrays vibrating in a first frequency and the cases of the array of micro-valve arrays vibrate in a second frequency, wherein the first frequency is different than the second frequency.

In some cases, the difference between the first frequency and the second frequency is determined as a function of physical properties of the mechanical connector.

In some cases, the apparatus further comprises a driver for generating an electronic signal and transmit the electronic signal to the vibrating element.

In some cases, the signal is a square wave. In some cases, the driver provides the vibrating element with a DC voltage, said DC voltage provides for bending the vibrating element, thereby changing a distance between the cases and the needles in the array of micro-valves.

In some cases, the apparatus further comprises a fluid reservoir that contains fluid, wherein the fluid is dispersed from the apparatus as mist. In some cases, the fluid flows from the fluid reservoir via the cases to the needles. In some cases, the fluid flows from the fluid reservoir via the needles to the cases. In some cases, the apparatus further comprises a pulling mechanism for pulling fluid from the fluid reservoir to the array of micro-valves. 11. The apparatus according to claim 10, wherein the pulling mechanism comprises a wick and an upper wick, said upper wick interacts with the array of micro-valves and is made of elastic material.

In some cases, the distance between a needle and a respective case of the array of micro-valves vary when the vibrating element vibrates the array of micro-valves. In some cases, the vibrating element is an ultrasonic piezoelectric element.

It is an object of the subject matter to disclose a method, comprising: receiving a user's selection of operation mode of an apparatus for outputting scent, said apparatus comprises a ultrasonic piezoelectric element for vibrating micro-valve cases and micro-valve needles; regulating an operation frequency of the apparatus for outputting scent according to the user's selection; wherein responsive to reception of a silent mode from the user of the apparatus, the ultrasonic piezoelectric element switches between an efficient nebulization frequency and a non- efficient nebulization frequency.

It is an object of the subject matter to disclose a method, comprising: providing an piezoelectric element with an electronic signal; the piezoelectric element is connected to an array of interconnected cases for outputting scented mist and to an array of interconnected needles; vibrating the array of interconnected needles and the array of interconnected cases according to the electronic signal; regulating the size of aperture between a case of the array of interconnected cases and a needle of the array of interconnected needles as a function of an amplitude of the electronic signal provided to the piezoelectric element.

### BRIEF DESCRIPTION OF THE DRAWINGS

Exemplary non-limited embodiments of the disclosed subject matter will be described, with reference to the following description of the embodiments, in conjunction with the figures. The figures are generally not shown to scale and any sizes are only meant to be exemplary and not necessarily limiting. Corresponding or like elements are optionally designated by the same numerals or letters.

FIG. 1A shows a micro-valve array for providing mist, according to exemplary embodiment of the subject matter;

FIG. 1B shows an apparatus for providing mist, according to exemplary embodiment of the subject matter;

FIG. 2A shows a bottom view of a micro-valve dispersion unit, according to exemplary embodiments of the subject matter;

FIG. 2B shows a top view of a micro-valve dispersion unit, according to exemplary embodiments of the subject matter;

FIG. 3A shows an upper cross section of a micro-valve dispersion unit, according to exemplary embodiments of the disclosed subject matter;

FIG. 3B shows a lower cross section of a micro-valve dispersion unit, according to exemplary embodiments of the disclosed subject matter;

FIG. 3C shows a base ring surrounding the cases, according to exemplary embodiments of the disclosed subject matter;

FIG. 4 shows a cross section of a micro-valve dispersion unit, according to exemplary embodiments of the disclosed subject matter;

FIG. 5A shows a piezoelectric element on a base ring, according to exemplary embodiments of the disclosed subject matter;

FIG. 5B shows a piezoelectric element provided with a DC signal, according to exemplary embodiments of the disclosed subject matter;

FIG. 6 shows an array micro valves that comprise needles connected to an cases, according to exemplary embodiments of the disclosed subject matter;

FIG. 7A shows an apparatus for dispersing mist, according to exemplary embodiments of the disclosed subject matter;

FIG. 7B shows a cross section of an apparatus for dispersing mist, according to exemplary embodiments of the disclosed subject matter;

FIG. 8 shows various signals transmitted from the driver to the piezoelectric element, according to exemplary embodiments of the disclosed subject matter; and,

FIG. 9 shows an apparatus for dispersing mist without a wick, according to exemplary embodiments of the disclosed subject matter.

### DETAILED DESCRIPTION

The subject matter discloses a method and system for producing scented mist using an ultrasonic device that comprises an array of micro valves. Each micro valve comprises a cases and a needle. The needles and the cases of the array of micro valves are connected by a mechanical connector which enables relative movement of needles relative to the case while the array of micro valves vibrates to disperse mist. The needles and cases move relative to each other on the same axis to open or close a volume via which the mist is dispersed. Movement of the needles and cases is generated by a vibrating mechanism, for example a piezoelectric element or an electromagnetic mechanism. One technical problem dealt with in the subject matter is to generate movement of the needles and cases in a dynamic manner, for example by generating a movement for the cases and a second movement for the needles. Another technical problem is to avoid noise generated by the system for producing scented mist when intermittently providing an electronic signal to the piezoelectric element that vibrates the plurality of cases and plurality of needles.

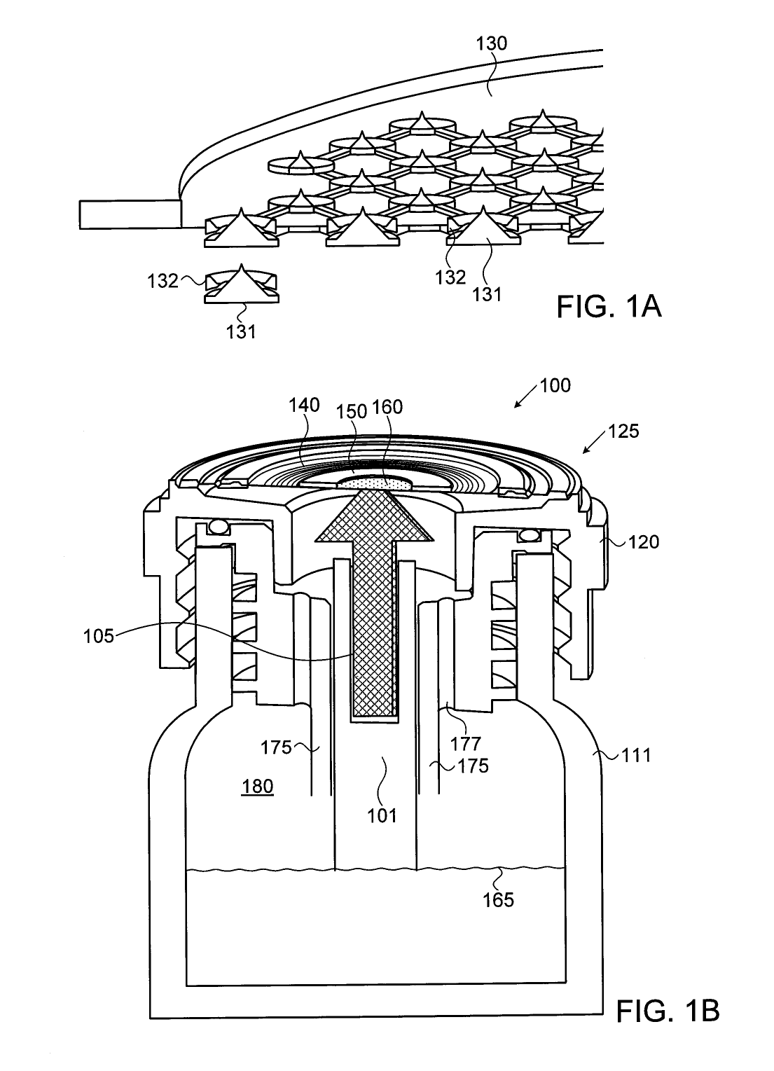

FIG. 1A shows a micro-valve Array, according to exemplary embodiment of the subject matter. The aforementioned array of micro-valves 130 comprises a plurality of micro valves. Each micro valve comprises a micro-valve case 132, and a micro-valve needle 131, each needle of the array of micro-valves 130 is engaged to a specific respective case. The arrangement of micro-valve cases and the arrangement of micro-valve needle are operationally connected such that said micro needles are inserted at least partially into said micro-valve cases. The distance between said micro-valve cases 132 and said micro-valve needles 131 is adjustable, so that the micro-valves can be opened and closed and adjusted in various sizes of an aperture between a micro-valve case and a micro-valve needle. Such adjustment is detailed below. The needle may close the case or open an aperture in various sizes, through which mist can flow before dispersed from the apparatus.

FIG. 1B shows an apparatus for providing scented, according to exemplary embodiment of the subject matter. The apparatus 100 comprises a container 180 that contains fluid 165 to be dispensed as mist by a micro-valve dispersion unit 160. The fluid 165 may be scented fluid. In some exemplary cases, the apparatus 100 may comprise more than one replaceable containers, each container comprises a different fluid.

The apparatus 100 further comprises a pulling mechanism 101 for pulling the fluid 165 towards the micro-valve dispersion unit 160. The pulling mechanism 101 may be a wick that pulls the fluid 165 upwards using capillary forces. The wick 101 may include two connected components—a base wick 101 and an upper wick 105. The upper wick 105 may be made of an elastic material to better interact with the micro-valve dispersion unit 160. The elastic material of the upper wick 105 may be a sponge-like material. The micro-valve dispersion unit 160 of the apparatus 100 is maneuvered by a vibrating element, for example a piezoelectric element 150. The wick 101 may be surrounded by a wick holder 175. The wick holder may be positioned near a vacuum prevention tube 177. The vacuum prevention tube 177 comprises apertures for allowing passage of air between the of area reverse nebulization reservoirs to the fluid reservoir 180, thereby preventing vacuum in the fluid reservoir 180. Such vacuum would prevent fluid to flow upwards using the wick 101.

The micro-valve dispersion unit 160 comprises an array of micro valves. Each micro valve of the array of micro valves comprises a case and a needle. The micro-valve dispersion unit 160 may comprises interconnected cases, as each case is associated with a specific needle. The piezoelectric element 150 is connected to array of micro valves. The needles and cases of each micro valve of the array of micro valves are connected via a mechanical connector, as detailed below. When the vibrating element, such as the piezoelectric element 150 vibrates, array of micro valves but the needles and cases vibrate differently due to a secondary movement enabled by the mechanical connector. The secondary movement may be a function of physical properties of the mechanical connector. The physical property may be length of the mechanical connector, strength of the material used to make the mechanical connector and the like. The relative movement between the needles and the cases of the array of micro valves allows for dispersion of mist via the cases.

The apparatus 100 may be divided into two separate units—a dispersion unit 120 and a reservoir unit 111. The reservoir unit 111 comprises the container 180 and is mechanically attached to the dispersion unit 120 that comprises the pulling mechanism 101 and the micro-valve dispersion unit 160. The dispersion unit 120 comprises a housing 125 for protecting the mechanisms and elements within the dispersion unit 120. The housing 125 is made of rigid material, such as plastics or metal and is configured to be hermetically sealed in order to prevent leakage of the fluid 165 from the apparatus 100 and allow only dispersion of mist via the micro-valve dispersion unit 160. As the array of micro valves of the micro-valve dispersion unit 160 vibrate and move when dispersing mist and the housing 125 is required to be sealed, there is a need to provide an element connecting the housing and the micro-valve dispersion unit 160. Such element is a bellows unit 140 positioned between the housing and the micro-valve dispersion unit 160. The bellows unit 140 move when the arrays of the micro-valve dispersion unit 160 but keeps the housing 125 firm. The bellows unit 140 may be made of elastic metal material, such as nickel. The bellows unit 140 may have a spring-like shape that enables the bellows unit 140 to be securely and firmly attached to the housing 125 on one end and be movable on a second end, as the second end is connected to the array of micro valves of the micro-valve dispersion unit 160. The spring-like shape and type of material of the bellows unit 140 also enables self-cleaning of scents from the bellows unit 140 when the arrays vibrate, utilizing the existing ultrasonic energy of the apparatus.

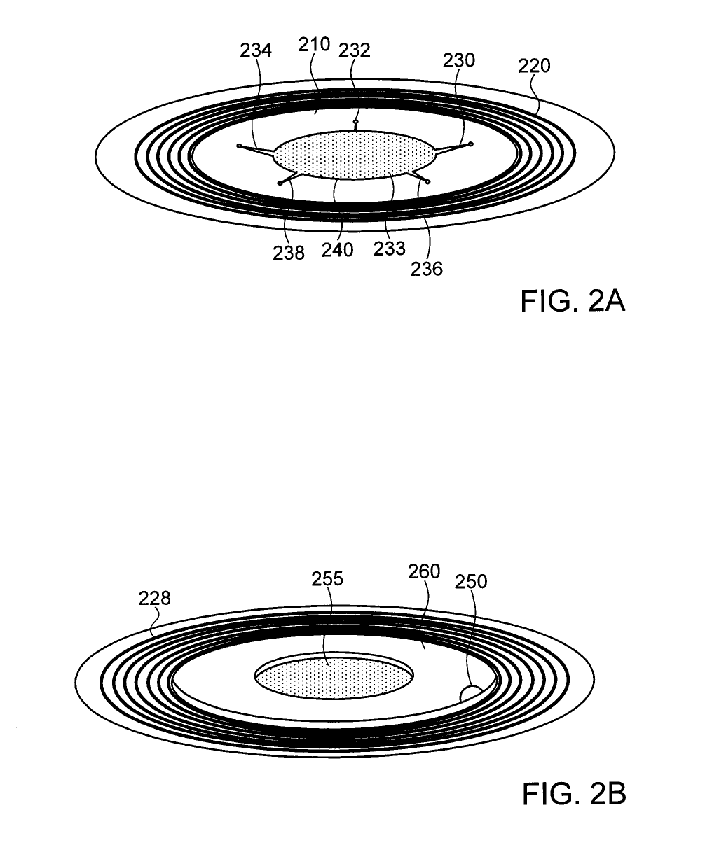

FIG. 2A shows a bottom view of a micro-valve dispersion unit, according to exemplary embodiments of the subject matter. The micro-valve dispersion unit comprises a base ring 210 configured to be attached to a vibrating element, for example a piezoelectric element. The base ring 210 surrounds a plurality of needles 240 that may be interconnected. The array of micro valves comprises needles 240, for example in a range of 10-6,000. Each of the needles 240 of the array of micro valves is associated with a respective case. The needles 240 may be positioned near a plurality of apertures 233 configured to allow fluid to enter a volume between the needles 240 and the cases of the array of micro valves. In some exemplary cases, the needles 240 are connected to the bottom portion of the base ring 210 using mechanical connectors 230, 232, 234, 236, 238. The mechanical connectors 230, 232, 234, 236, 238 may be elastic or flexible to allow relative movement of the needles 240, relative to the cases. The base ring 210 may be surrounded by a bellows unit 228 as disclosed above.

FIG. 2B shows a bottom view of a micro-valve dispersion unit, according to exemplary embodiments of the subject matter. The micro-valve dispersion unit comprises cases 255 via which the mist is dispersed. Each needle of the array of micro valves is associated with a specific respective case. Movement of the needles and cases regulates the size of the aperture via which the mist is dispersed.

The micro-valve dispersion unit also comprises a vibrating element, for example a piezoelectric element 260 attached to the base ring 210 of FIG. 2A. In some cases, the base ring 210 is connected on one surface to the vibrating element 260 and on another surface to the mechanical connectors used to connect the base ring 210 with the cases or needles. Said attachment may be using glue, hot welding, cold welding and the like. The piezoelectric element 260 receives an electronic signal from a signal generator, such as an electronic driver. The piezoelectric element 260 vibrates the array of micro valves. Such vibration is dynamic. Prior art mist dispersion devices first generated an aperture between the cases and needles and then vibrated the needles and cases. The apparatus of the disclosed subject matter vibrates the array of micro valves that comprises needles 240 and cases 255 and utilizes the mechanical connectors 230, 232, 234, 236, 238 to achieve apertures in variable size during the vibration. The mechanical connectors 230, 232, 234, 236, 238 enable secondary movement of the needles 240 relative to the cases 255. The vibration of the subject matter is used to regulate the size of the apertures between each pair of a needle and a case, not just to cause mist to be outputted via the case.

The piezoelectric element 260 comprises two electrodes—one electrode connected to the upper section and another electrode connected to the bottom section. In some exemplary cases, the electrode 250 connected to the bottom section is wrapped around the piezoelectric element 260 to be able to be connected to the signal generator with the first electrode. The micro-valve dispersion unit also comprises a bellows unit 228 connecting the base ring 210 to the housing of the apparatus.

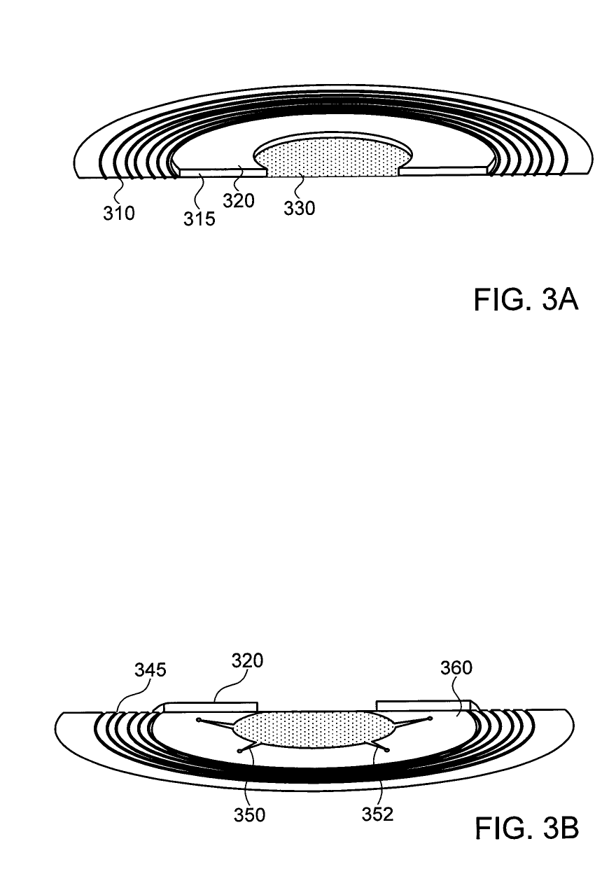

FIG. 3A shows an upper cross section of a micro-valve dispersion unit, according to exemplary embodiments of the disclosed subject matter. The upper cross section comprises a bellows unit 310. The bellows unit 310 has a cross sectional shape of a spring. The bellows unit 310 is positioned between the housing of the apparatus and the base ring on which the piezoelectric element is located. FIG. 3A shows an upper section 320 of the piezoelectric element and a cross section 315 of the piezoelectric element. The piezoelectric element is ring shaped, located on a ring base that surrounds the cases 330.

FIG. 3B shows a lower cross section of a micro-valve dispersion unit, according to exemplary embodiments of the disclosed subject matter. The lower cross section shows a spring shaped bellow 345 surrounding a base ring 360. The base ring 360 may be connected to the needles via mechanical connectors 350, 352.

FIG. 3C shows a base ring 370 surrounding the cases 380, according to exemplary embodiments of the disclosed subject matter.

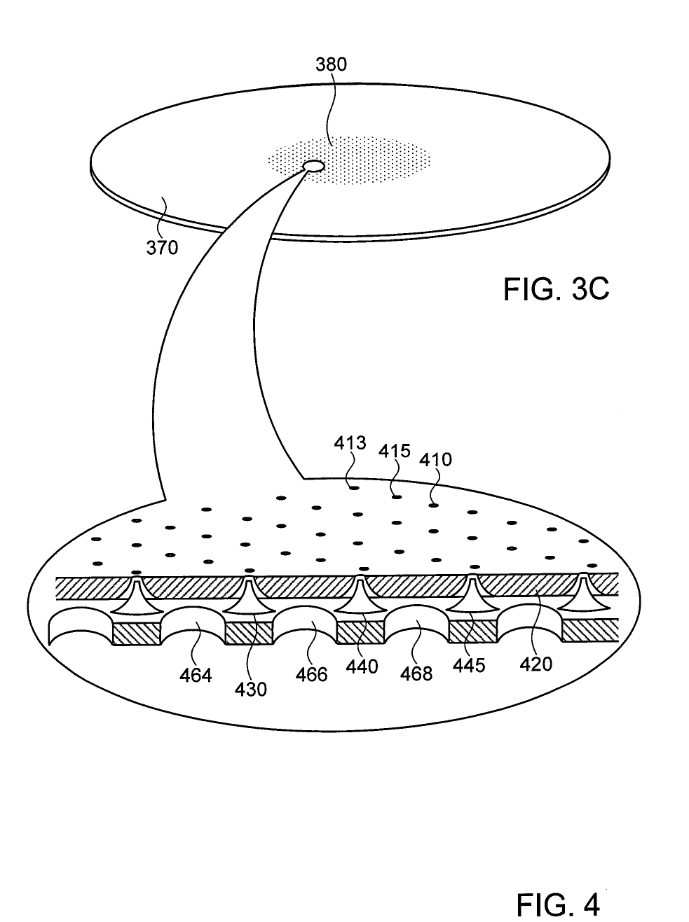

FIG. 4 shows a cross section of a micro-valve dispersion unit, according to exemplary embodiments of the disclosed subject matter. The micro-valve dispersion unit comprises pairs of needles and cases. Mist dispersed using the array of micro-valve dispersion unit may be dispersed from the needles via the cases and then outwards, or from the cases via the needles and then outwards. The needles comprises a plurality of needles, such as needles 430, 440 and 445. Each needle of the plurality of needles of is connected to a specific respective case 410. Other cases include cases 413 and 415. The needles are located adjacent to a set of apertures 464, 466, 468 via which the liquid can flow from the container 160 towards a dispersion volume 420. The dispersion volume 420 is a volume between the needles and the cases 410. In some exemplary cases, such dispersion volume 420 may be defined by a volume between pairs of respective needle and case. In some cases, the liquid flows from the container 160 via the cases 410 to the dispersion volume 420, then dispersed via the needles to the air. In such a case, the set of apertures 464, 466, 468 is positioned near the cases 410 and the needles area is hermetically sealed.

The needles and the cases 410 move according to vibrations caused by the vibrating element, such as a piezoelectric element. The frequency of movement of the needle and case of each micro valve is determined according to the signal provided to the piezoelectric element. The needles vibrates in a first frequency and the cases vibrates in a second frequency. The first frequency is enabled to be different than the second frequency according to a secondary movement enabled by the mechanical connectors. The physical properties of the mechanical connector may effect the movement of the needles, for example in terms of amplitude and frequency. For example, the elasticity of the mechanical connectors may result in lower amplitude of the movement of the needles than the amplitude of the movement of cases.

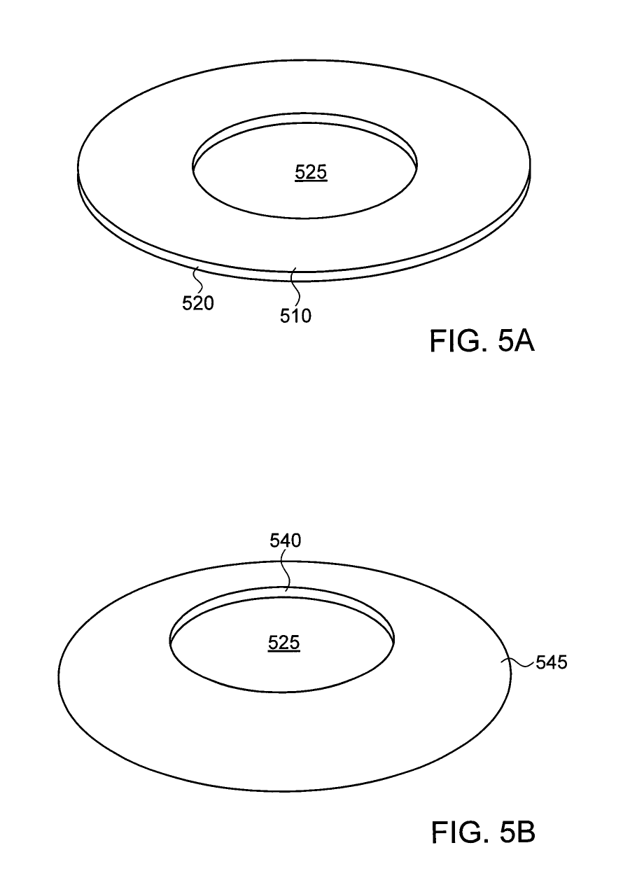

FIG. 5A shows a piezoelectric element on a base ring, according to exemplary embodiments of the disclosed subject matter. FIG. 5A shows an upper section 510 and a cross section 520 of the piezoelectric element and an upper view of the cases 525.

FIG. 5B shows a piezoelectric element provided with a DC signal, according to exemplary embodiments of the disclosed subject matter. When induced with a DC voltage signal from the driver, the piezoelectric element bends its shape. The height of the piezoelectric element increases as a result of the DC signal. The height may be defined by the distance between the upper section 540 and the cases 525.

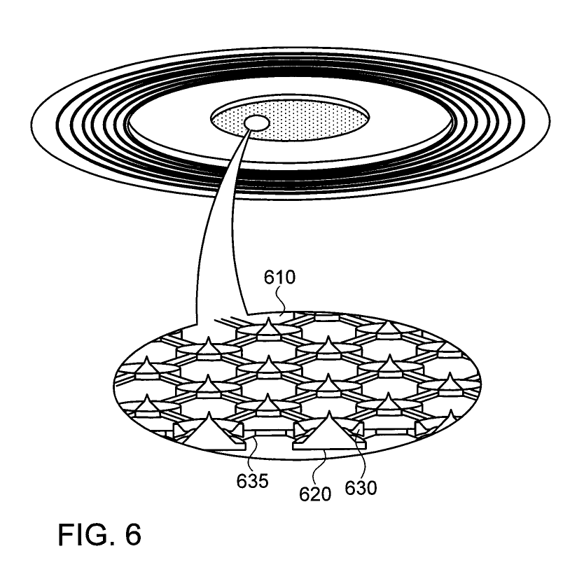

FIG. 6 shows needles connected to cases 610, according to exemplary embodiments of the disclosed subject matter. The needle 620 is configured to be inserted into a respective specific case 630. The specific case 630 may be of a conic shape, to fit to the upper section of the needle 620. The needles may be interconnected using a connection structure 635 made of nickel, the structure 635 contains open zones or a set of apertures located between needles on the structure 635 to allow liquid flow from the container towards the micro-valve cases. Each needle has a respective case. The movement of the arrays is limited to substantially upwards and downwards, towards and against each surface. The movement of the arrays is limited to prevent a case in which the entire needle is outside the case. That is, when a needle is farthest from the case, to allow flow of a mist there-between, the needle cannot be associated with another case. When the needles are positioned nearby or surrounded by a set of apertures, the area of the cases is hermetically sealed between the cases.

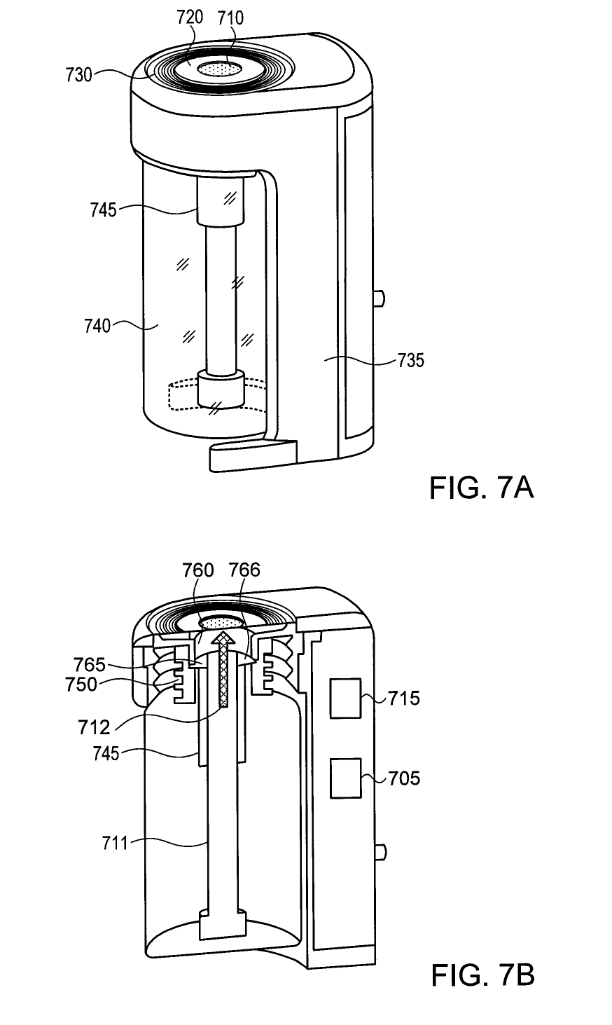

FIG. 7A shows an apparatus for dispersing mist, according to exemplary embodiments of the disclosed subject matter. The apparatus comprises a micro-valve dispersion unit and a reservoir unit. The micro-valve dispersion unit comprises a bellows unit 730 surrounding a base ring (not shown), on which a piezoelectric element 720 is attached. The base ring surrounds the cases 710. In some exemplary cases of the subject matter, each case of the cases 710 is connected to a needle. Each pair of case and needle may also be defined as a micro-valve that regulates flow of mist in a volume between the needle and the case.

The reservoir unit comprises a housing and a wick housing 745. The wick 711 is configured to allow passage of fluid from the fluid container 740 to towards the micro-valve dispersion unit. The wick 711 is connected to an upper wick 712 that interacts with the micro-valve dispersion unit.

FIG. 7B shows a cross section of an apparatus for dispersing mist, according to exemplary embodiments of the disclosed subject matter. The apparatus comprises a driver 715 configured to generate electronic signals and inject the vibrating element, such as piezoelectric element 720 with the generated signal. The signals generated by the driver 715 may cause the piezoelectric element 720 to vibrate the array of micro valves that comprises a plurality of pairs of needles and cases. The needles and cases of the array of micro valves may move in a different manner since they are connected via an elastic mechanical connector. The apparatus also comprises a power source 705 configured to provide power to the electronic units of the apparatus, such as the driver 715.

The apparatus further comprises a wick 712 configured to allow passage of fluid from the fluid container 740 to towards the micro-valve dispersion unit. The wick 712 is in contact with the micro-valve dispersion unit, for example with the needles. In some cases, when the cases is located between the fluid reservoir and the needles, the cases are in contact with the wick 712.

The apparatus further comprises a screw housing 750 allowing for connecting a housing to the micro-valve dispersion unit. The screw housing 750 may be located at the external wall of the micro-valve dispersion unit.

The apparatus further comprises reverse nebulization reservoirs 765, 766. It is herein acknowledged that some of the mist forms internally to the emitter during mist production. The problem is especially manifest with high viscosity fluids, which have a tendency to accumulate, damage and block below the nebulization system during operation of the unit especially when high viscous liquids are used. According to the subject matter, fluid is accumulated at the area 760 in which the wick 712 is in contract with the micro-valve dispersion unit. The fluid then flows downwards to the reverse nebulization reservoirs 765, 766, which direct the fluids which were nebulized downwards towards the wick 712. This way, the apparatus avoids blockage of the micro-valve dispersion unit.

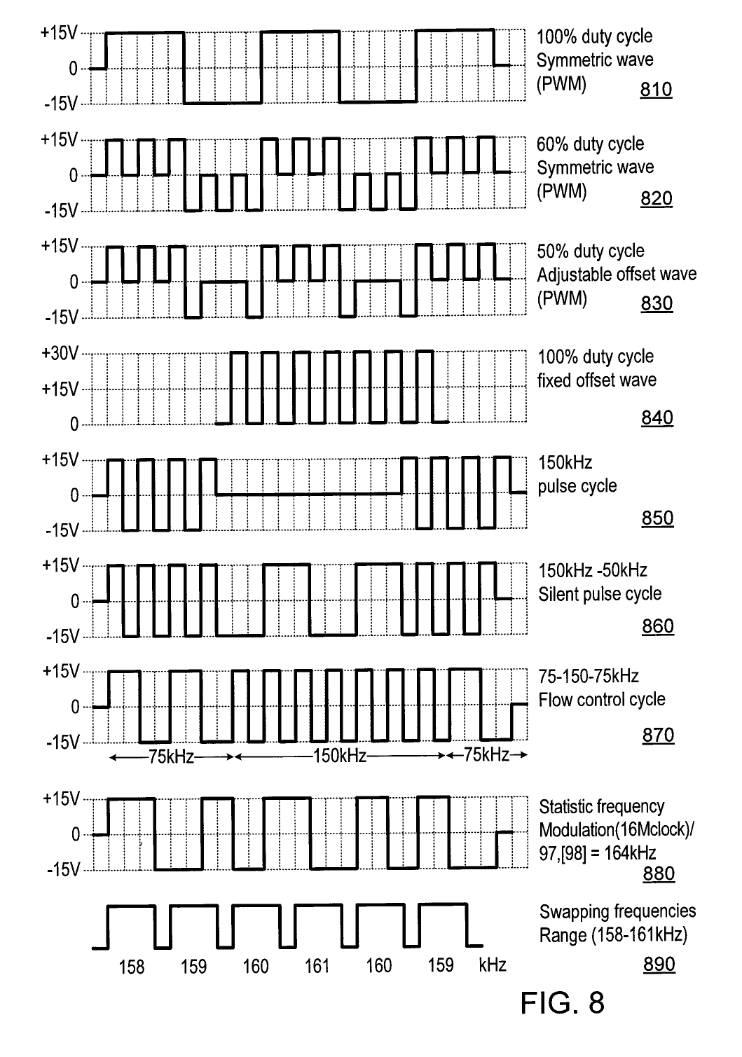

FIG. 8 shows various signals transmitted from the driver to the piezoelectric element, according to exemplary embodiments of the disclosed subject matter. The X axis of signals 810-860 denotes time, while the X axis in signals 870-890 denotes frequency. The Y axis denotes voltage. The signal 810 shows a 100% duty cycle symmetric wave that provides for a pulse width modulation (PWM), in which the frequency of the signal is modulated every predetermined period of time. The modulation provided by the signal 810 provides for vibrating of the piezoelectric element, which results in vibrating the micro-valve array and momentarily changing the distance between each needle and case of the micro-valve array.

The signal 820 discloses performing a PWM as in the signal 810, by providing less energy in the signal. The signal 820 discloses a duty cycle that comprises zero voltage, which reduces energy consumption by the apparatus. The zero voltage provided to the piezoelectric element reduces energy consumption of the piezoelectric element by 40-60%, according to the duration of zero voltage.

The signal 830 discloses a DC duty cycle with adjustable offset wave. In every cycle, the signal 830 comprises 3 positive voltages and two negative voltages with a zero voltage with a duration about double than the duration of each of the negative voltages. Appling DC off-set provides for changing the shape of the piezoelectric element, as shown in FIG. 5B. Changing the shape of the piezoelectric element comprises changing the distance between the upper point and the lowest point of the piezoelectric element, which results in changing the distance between each needle and case of the micro-valve array.

The voltage 840 discloses a 100% duty cycle with an offset. As the regular mode provides for a duty cycle of 15 volts, the signal 840 provides for a voltage of 30 volts.

The voltage 850 discloses a pulse cycle for preventing dispersed mist to intervene with previously dispersed mist. The apparatus of the disclosed subject matter emits mists upwards. Then, the mist falls downwards. The pulse cycle 850 prevents the collision of the falling mist with the newly generated mist that is dispersed upwards. The pulse cycle results in mist dispersion for a predefined period of time, and then termination of dispersion. For example, dispersing mist for 10 ms and ceasing dispersion for 30 ms.

The voltage 860 discloses a silent pulse cycle. The pulse cycle provides for intermittent pulses after constant voltage. The constant voltage is at inefficient nebulization frequencies. Then, the driver keeps transmitting a signal to the piezoelectric element, but the nebulization stops until the signal is back to the efficient nebulization frequency.

The voltage 870 discloses a flow control signal. The flow control provides for transmitting a signal in a low frequency and high frequency on an intermittent manner. For example, the driver transmits a signal of 75 kHz for 50 ms, and then transmits a signal of 150 kHz for another 50 ms. A lower frequency results in higher amplitude between a needle and a case in a respective micro-valve array. As such, operating in 75 kHz allows passage of fluid to the volume between the needle and the case. Then, the piezoelectric element switches to operate in another frequency, such as 150 kHz, which is known to be more efficient in nebulizing the mist from the apparatus. This way, the apparatus disperses mist when the driver generates a signal of 150 kHz and accumulates mist between the needle and case when the driver generates a signal of 75 kHz. The voltage 880 discloses defining the optimal frequency in low-consumption clocks, such as 16M clock. For example, the 16M clock obtains data that the preferred harmony of the needles and the cases is 164 kHz. The preferred harmony may be a function of physical properties of the arrays, and the mechanical connector. Then, the clock wishes to switch operation mode every 97.5 clock units. However, the clock cannot count 97.5 units and switches between operation modes every 98 clock units and then switches between operation modes every 98 clock units.

The voltage 890 discloses scanning frequencies on a frequency band to determine the optimal frequency for mist dispersion. The driver generates a signal in frequencies that change in time. First 158 kHz, then 159 kHz, then 160 kHz and then 161 kHz. When the driver reaches a known maximum frequency, it transmits signals in decreasing frequencies, to detect electric current flow of the piezoelectric element. Alternatively, the driver continues to swap on the range of frequencies without searching for optimal frequency. In another alternative case, the driver obtains predefined frequencies that are optimal for the mechanical properties of the needles and the cases.

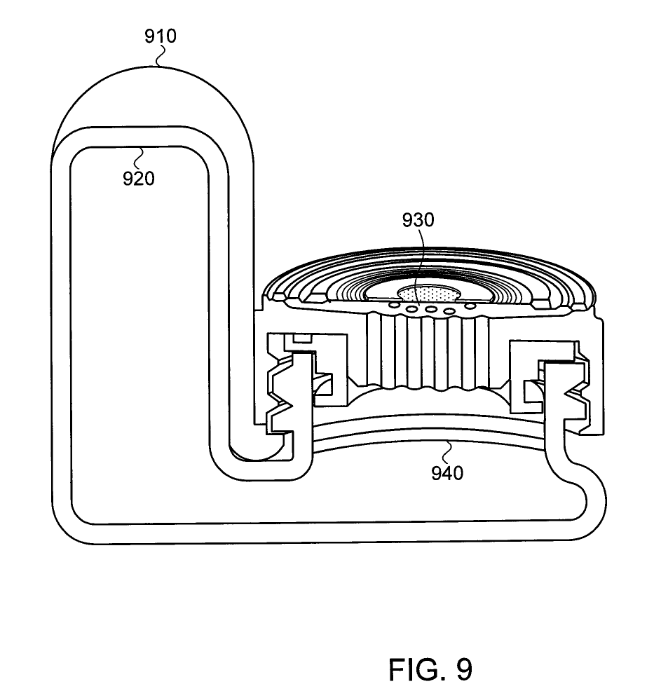

FIG. 9 shows an apparatus for dispersing mist without a wick, according to exemplary embodiments of the disclosed subject matter. The apparatus comprises a u-shaped container. One top 910 of the u-shaped container includes the fluid 920 contained by the u-shaped container. Another top of the u-shaped container contains the ultrasonic micro-valve array 930 from which the mist is dispersed. The two tops make the wick unnecessary, as the fluid flows to the lower end 940 of the micro-valve array 930 using communicating vessels law.

While the disclosure has been described with reference to exemplary embodiments, it will be understood by those skilled in the art that various changes may be made and equivalents may be substituted for elements thereof without departing from the scope of the subject matter. In addition, many modifications may be made to adapt a particular situation or material to the teachings without departing from the essential scope thereof. Therefore, it is intended that the disclosed subject matter not be limited to the particular embodiment disclosed as the best mode contemplated for carrying out this subject matter, but only by the claims that follow.

1. An apparatus, comprising:
an array of micro-valves for dispersing mist, each micro valve of the array of micro-valve comprises a needle and a case;
an ultrasonic vibrating element for vibrating the array of micro-valves;
a mechanical connector for connecting the needles and cases of the array of micro-valves, thus enabling generation of a secondary movement of the needles relative to the cases while the array of micro-valves vibrates, said secondary movement is generated by the vibration of the ultrasonic vibrating element.

2. The apparatus according to claim 1, wherein the mechanical connector results in the needles of the array of micro-valves vibrating in a first frequency and the cases of the array of micro-valves vibrate in a second frequency, wherein the first frequency is different than the second frequency.

3. The apparatus according to claim 2, wherein the difference between the first frequency and the second frequency is determined as a function of physical properties of the mechanical connector.

4. The apparatus according to claim 1, further comprises a driver for generating an electronic signal and transmit the electronic signal to the vibrating element.

5. The apparatus according to claim 4, wherein the signal is a square wave.

6. The apparatus according to claim 4, wherein the driver provides the vibrating element with a DC voltage, said DC voltage provides for bending the vibrating element, thereby changing a distance between the cases and the needles in the array of micro-valves.

7. The apparatus according to claim 1, further comprises a fluid reservoir that contains fluid, wherein the fluid is dispersed from the apparatus as mist.

8. The apparatus according to claim 7, wherein the fluid flows from the fluid reservoir via the cases to the needles.

9. The apparatus according to claim 7, wherein the fluid flows from the fluid reservoir via the needles to the cases.

10. The apparatus according to claim 7, further comprises a pulling mechanism for pulling fluid from the fluid reservoir to the array of micro-valves.

11. The apparatus according to claim 10, wherein the pulling mechanism comprises a wick and an upper wick, said upper wick interacts with the array of micro-valves and is made of elastic material.

12. The apparatus according to claim 1, wherein a distance between a needle and a respective case of the array of micro-valves vary when the vibrating element vibrates the array of micro-valves.

13. The apparatus according to claim 1, wherein the vibrating element is an ultrasonic piezoelectric element.

14. A method, comprising:
receiving a user's selection of operation mode of an apparatus for outputting scent, said apparatus comprises a ultrasonic vibrating an array of micro-valves;
regulating an operation frequency of the apparatus for outputting scent according to the user's selection;
wherein responsive to reception of a silent mode from the user of the apparatus, the ultrasonic vibrating element switches between an efficient nebulization frequency and a non-efficient nebulization frequency.

15. A method, comprising:
providing a vibrating element with an electronic signal;
the vibrating element is connected to an array of micro valves for outputting mist;
vibrating the array of array of micro valves according to the electronic signal;
regulating the size of aperture between a case of the array of interconnected cases and a needle of the array of interconnected needles as a function of an amplitude of the electronic signal provided to the vibrating element.
